SEQUENCE LISTING (3 sequences)

SEQ ID NO: 1 - DNA, 228 bp, synthetic construct
  source 1..228: mol_type = other DNA
ttcaacggtgtggactttgacatggtgggtcgtggcaccggcaatccaaatgatggttatgaggagttaaacctgaagtccaccaagggtgccctccagttctccccctggattctggtccctcaaatcgggtatggcttccatcagtacctgcccttccccgacgggatgtcgcctttccaggccgccatgaaagatggctccggataccaagtccatcgcacaatg

SEQ ID NO: 2 - DNA, 15 bp, synthetic construct
  source 1..15: mol_type = other DNA
ttcaacggtgtggac

SEQ ID NO: 3 - DNA, 15 bp, synthetic construct
  source 1..15: mol_type = other DNA
cattgtgcgatggac

(19) United States
(12) Patent Application Publication
(10) Pub. No.: US 2024/0002909 A1
(43) Pub. Date: Jan. 4, 2024

(54) USE OF ENZYMES FOR ALTERING RATIOS OF PARTIALLY MATCHED POLYNUCLEOTIDES

(71) Applicant: ALLELE BIOTECHNOLOGY AND PHARMACEUTICALS, INC., San Diego, CA (US)

(72) Inventors: Jiwu WANG, La Jolla, CA (US); Jianhua DING, San Diego, CA (US)

(21) Appl. No.: 18/212,605

(22) Filed: Jun. 21, 2023

Related U.S. Application Data
(63) Continuation of application No. 16/532,396, filed on Aug. 5, 2019.
Application No. 16/532,396 is a continuation of application No. 13/136,016, filed on Jul. 19, 2011, now abandoned.
(60) Provisional application No. 61/365,374, filed on Jul. 19, 2010.

(51) Int. Cl. C12Q 1/6816

(52) U.S. Cl. CPC C12Q 1/6816

(57) ABSTRACT

The present disclosure relates to novel methods of discriminating and/or detecting mis-matched polynucleotide populations in a sample by determining the ratios of mismatched polynucleotide species after specific enzymatic digestion treatment. Aspects of this disclosure includes obtaining, enhancing and/or determining the amount of one DNA or RNA species versus another in a given sample following enzyme digestion treatment; determining the relative abundance of the species contained in the sample based on the changes in the relative ratios following enzymatic treatment.

### RELATED APPLICATION

This application is a continuation of U.S. application Ser. No. 16/532,396, filed Aug. 5, 2019, which was a continuation of U.S. application Ser. No. 13/136,016, filed Jul. 19, 2011, which claims the benefit of priority to U.S. provisional application Ser. No. 61/365,374, filed on Jul. 19, 2010, the contents of which is incorporated by reference in its entirety.

### FIELD OF THE INVENTION

The present disclosure relates to novel methods of discriminating and/or detecting mis-matched polynucleotide populations in a sample by determining the ratios of mismatched polynucleotide species after specific enzymatic digestion treatment. More specifically, certain aspects of this disclosure relates to obtaining, enhancing and/or determining the amount of one DNA or RNA species versus another in a given sample following enzyme digestion treatment; determining the relative abundance of the species contained in the sample based on the changes in the relative ratios following enzymatic treatment.

### SEQUENCE LISTING

The instant application contains a Sequence Listing that has been submitted electronically in XML format and is hereby incorporated by reference in its entirety. Said XML copy, created on Jun. 21, 2023, is named F6035-00181_ST26.xml and is 3,995 bytes in size.

### BACKGROUND

The amount of genetic materials is highly regulated in the cells of all species. The expression of genetic information from chromosomes and DNA is highly controlled so that the cell can function in a balanced fashion. The disruption of the balance may have deleterious consequences leading to diseases and/or disorders. Although there are numerous causes that can induce the deregulation of genetic information, the key central carriers of these information are relatively simple—chromosomes, DNA and the gene expression patterns reflected in the RNA profiles. It is well-documented that copy number changes of whole or partial chromosome; mutation of nucleotides; and mRNA isoform ratio variation are the major contributors of the deregulation of genetic information. Therefore, detection of such genetic variation is critical for the diagnosis and determination of onset and development of disease as well as providing means of monitoring course of disease and/or correct therapy or treatment.

Various methodologies have been developed to identify genetic variations in basic and clinical biological research, e.g. PCR, MASS analysis, DNA microarray, and sequencing. However, in certain conditions, the ratio variation of genetic materials is below the level of detection that these and other commonly used methods can not be directly applied for their intended purposes.

Certain approaches have been considered to increase the probability of detecting minor polynucleotide species, especially when a limited amount of samples is available, such as for example, maternal blood in neonatal diagnosis applications. These include digital PCR [4, 5], microfluidics digital PCR [2], temperature switch PCR [6], multiplex ligation-dependent probe amplification (MLPA) [7]. However, these methods often require extensive PCR reactions (e.g. in digital PCR), or involve complicated multiple steps for improving sensitivity. In addition, many of the methods in existence have not been validated for general application or clinical use.

### SUMMARY OF THE INVENTION

Accordingly, in view of the problems associated with the previously known procedures, improved methods useful for sensitive detection of low level DNA or RNA signals or signal ratios or ratio variations are desired. The present disclosure is directed to the unexpected and surprising discovery that comparisons of ratios of polynucleotide species, including mis-matched species, in a sample following enzymatic treatment, enabled determination/detections of low level polynucleotide variations in a sample. In addition, the ratio comparisons methods in the instant disclosure allowed for enhanced accuracy in determining the relative percentage or ratio of DNA alleles or RNA isoforms in sample pools.

In one aspect, a method is provided A method for calculating the ratio of nucleic acids in a region with or without mismatched portions, said method comprising: a) denaturing the double-stranded nucleic acids that are of different identities but have homologous sequences; 2) reannealing the resulting single-stranded nucleic acids to form either homoduplex or heteroduplex; c) contacting said duplex nucleic acids with an enzyme which cleaves mismatches in duplex nucleic acids; and d) detecting the presence of the surviving homoduplex nucleic acids spanning the region that is the target of the enzyme action thereby increase the ratio of the minor species of nucleic acids.

In one embodiment, said enzyme is a bacteriophage or a eukaryotic enzyme. In another embodiments, the bacteriophage enzyme is T4 Endonuclease, or T7 Endonuclease I. In another embodiment, the enzyme is lambda endonuclease.

In another aspect, the one strand of duplex nucleic acid is obtained from a eukaryotic cell, a eubacterial cell, a bacterial cell, a mycobacterial cell, a bacteriophage, a DNA virus, or an RNA virus. In one embodiment, the strand of said duplex nucleic acid is obtained from a human cell. In yet another embodiment, the duplex nucleic acid comprises at least one strand having a wild-type sequence.

In certain other aspect, the detection of a mismatch indicates the presence of a mutation. In one embodiment, the mutation is diagnostic of a disease or condition.

In yet another aspect, a method is provided for calculating the ratio of nucleic acids in a region with or without mismatched portions, said method comprising: a) denaturing the double-stranded nucleic acids that are of different identities but have homologous sequences; b) reannealing the resulting single-stranded nucleic acids to form either homoduplex or heteroduplex; c) contacting said duplex nucleic acids with an enzyme which cleaves mismatches in duplex nucleic acids; and d) detecting the presence of the surviving homoduplex nucleic acids spanning the region that is the target of the enzyme action thereby increase the ratio of the minor species of nucleic acids; e) determining the relative amounts of matched and mismatched species in the sample.

In yet another aspect, a method of enhancing pairing of DNA fragment after enzymatic digestion in a sample wherein the method comprises addition of T7 endonuclease I is provided.

### BRIEF DESCRIPTION OF THE DRAWINGS

The invention will now be described in relation to the drawings in which:

FIG. 1 shows the annealing patterns between DNA strands containing varied nucleotide. Light color bars represent varied nucleotides between DNA strands.

FIG. 2 shows PCR amplified LanY gene fragments. 5 ul PCR products were loaded in 1.5% agarose gel.

FIG. 3 show an exemplary T7 endonuclease I digestion at 20 C. 25 ng of DNA was used for each reaction in 10 ul. 12.5 ng of wild type and 12.5 ng mutant DNA were used in the mixed reactions (lanes 7-12). DNA was mixed in T7 endonuclease I buffer and denatured (94 C, 5 min) and re-nature (60 C, 1 min). Renatured DNA was cooled to 5 C and 1 ul (1 U/ul) T7 endonuclease I was added to each reaction. Reaction mixtures were incubated at 20 C for 30 minutes. Digestion was checked in 1.5% agarose.

FIG. 4 shows exemplary T7 endonuclease I digestion at 25 C for 2 hours. In the homologous DNA controls, 31.5 ng wild type DNA and 39.6 ng mutant DNA was used, respectively. In heteroduplex test, 15.7 ng wild type and 19.8 ng mutant DNA were used. DNA was denatured at 94 C and annealed at 60 degree C. T7 endonuclease I was added after DNA mixture was cooled to 5 C. Digested DNA was checked in 1.5% agarose gel.

FIG. 5 shows exemplary T4RNase H digest on blunt or recessive 5′ ends. DNA oligos of the same length (left) or different lengths (right) were labeled with P32 on the 5′ end, annealed, and treated with T4RNase H. The blunt end (left) or recessive ends (bottom strands, right) are cut by the enzyme, whereas the overhanging 5′ end (top strand, right) is not recognized by T4RNase H.

### DETAILED DESCRIPTION OF THE INVENTION

All terms not defined herein have their common meanings recognized in the art. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention.

The ability to detect mismatches in coding and non-coding DNA, as well as RNA, is important in a number of diagnostic and therapeutic applications. Mismatch may occur at a single nucleotide or over multiple nucleotides, and may result from a frame shift, stop codon, or substitution in a gene, each of which can independently render an encoded protein inactive. Alternatively, the mismatch may indicate a genetic variant which is harmless, resulting in a protein product with no detectable change in function (for example, gene polymorphism). Single base mismatches can include G:A, C:T, C:C, G:G, A:A, T:T, C:A, and G:T, with U being substituted for T when the nucleic acid strand is RNA. Nucleic acid loops can form when at least one strand of a mismatch-containing sequence, or heteroduplex, includes a deletion, substitution, insertion, transposition, or inversion of DNA or RNA.

In one aspect, mismatch detection may be used for identifying or evaluating mutations in nucleic acid sequences. Mutations are heritable changes in the sequence of the genetic material of an organism which can cause fatal defects like hereditary diseases or disorders. As a result, methods for mutation detection are important in medical diagnostics. Although mutations can be localized with great precision by DNA sequencing (Sanger et al. Proc. Natl. Acad. Sci. USA 74: 5463-5467 (1977)), the procedure is relatively time consuming and expensive, and requires toxic chemicals.

As used herein, the term “mismatch” includes that a nucleotide in one strand of DNA or RNA does not or cannot pair through Watson-Crick base pairing and n-stacking interactions with a nucleotide in an opposing complementary DNA or RNA strand. Thus, adenine in one strand of DNA or RNA would form a mismatch with adenine in an opposing complementary DNA or RNA strand. Mismatches occur where a first nucleotide cannot pair with a second nucleotide in an opposing complementary DNA or RNA strand because the second nucleotide is absent (i.e., one or more nucleotides are inserted or deleted). The methods of the instant disclosure are especially useful in detecting a mismatch in a test nucleic acid which occurs in low abundance in a sample.

As used herein, an “enzyme” is any protein capable of recognizing and cleaving a cruciform DNA as well as any mismatch (for example, a mismatch loop) in a heteroduplex template. Exemplary enzymes include, without limitation, T4 endonuclease VII, Saccharomyces cerevisiae Endo X1, Endo X2, Endo X3, and CCE1, T7 endonuclease I, E. coli MutY. mammalian thymine glycosylase, topoisomerase I from human thymus and deoxyinosine 3′ endonuclease. In a given mismatch detection assay, one or several enzymes can be used.

A “mutation,” as used herein, refers to a nucleotide sequence change (i.e., a single or multiple nucleotide substitution, deletion, or insertion) in a nucleic acid sequence that produces a phenotypic result. A nucleotide sequence change that does not produce a detectable phenotypic result can be a “polymorphism.”

As used herein, the term “heteroduplex” is meant a structure formed between two annealed, complementary nucleic acid strands (e.g., the annealed strands of test and reference nucleic acids) in which one or more nucleotides in the first strand are unable to appropriately base pair with those in the second opposing, complementary strand because of one or more mismatches. Examples of different types of heteroduplexes include those which exhibit an exchange of one or several nucleotides, and insertion or deletion mutations.

The term “complementary,” as used herein, means that two nucleic acids, e.g., DNA or RNA, contain a series of consecutive nucleotides which are capable of forming matched Watson-Crick base pairs to produce a region of double-strandedness. Thus, adenine in one strand of DNA or RNA pairs with thymine in an opposing complementary DNA strand or with uracil in an opposing complementary RNA strand. The region of pairing is referred to as a “duplex.” A duplex may be either a homoduplex or a heteroduplex.

The methods described herein are useful for detecting DNA mutations associated with mammalian diseases (such as cancer and various inherited diseases), as well as mutations which facilitate the development of therapeutics for their treatment. Alternatively, the methods are also useful for forensic applications or the identification of useful traits in commercial (for example, agricultural) species.

Those skilled in the art will recognize that the disclosure is also useful for other purposes. For example, the claimed method facilitates detection of single base pair mismatches in cloned DNA, for example, mutations introduced during experimental manipulations (e.g., transformation, mutagenesis, PCR amplification, or after prolonged storage or freeze:thaw cycles). This method is therefore useful for testing genetic constructs that express therapeutic proteins or that are introduced into a patient for gene therapy purposes.

The disclosure generally relates to novel methods of discriminating and/or detecting mis-matched polynucleotide populations in a sample by determining the ratios of mismatched polynucleotide species after specific enzymatic digestion treatment. More specifically, certain aspects of this disclosure relates to obtaining, enhancing and/or determining the ratios of the amount of one DNA or RNA species versus another in a given sample following enzyme digestion treatment; determining the relative abundance of the species contained therein based on the ratios. The disclosure also provides methods for reducing or removing target species through matching/digesting.

As used herein, single nucleotide mismatch, multiple point mismatches, or difference in length even when the matching regions have perfect base pairing can all be considered as partial matching. The change of DNA or RNA ratios of partially matched, homologues sequences may be used for enhancing diagnosis, as tools for cellular and molecular biology experiments, or as means to remove disease-related DNA or RNA species for therapy.

The disclosure generally provides methods based on preferential digest by the activities of nucleases that can recognize and remove specific species of DNA or RNA from a population within a given sample. The embodiments in the present disclosure differ from all previously known technologies in that the ratio of polynucleotide species is altered by the disclosed methods and relied upon for detection and analysis. Utility of the method can be found in virtually all areas that involve detection or profiling of DNA or RNA signals in basic biological research or clinical diagnosis.

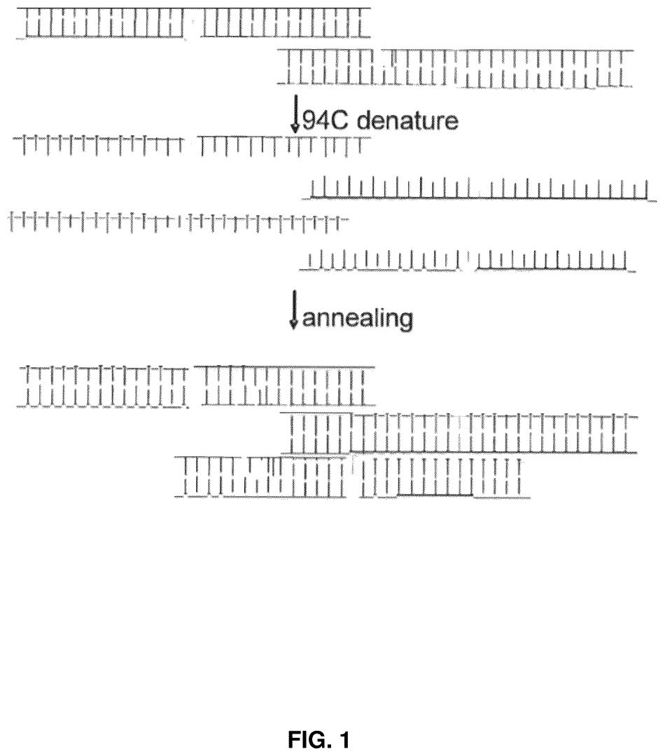

In one aspect, the current invention provides a method of discriminating two DNA species based on one or more mismatched basepairs. DNA double helix is formed by two complementary single strands of DNA via nucleotide base paring. DNA double strands can be separated when heated at temperature higher than melting temperature (Tm) and the separated DNA strands can form double helix at or below Tm. Free energy (dG) of matched base pairing is between −0.9 to −3.4 kcal/mol. One mismatched base pair in double stranded DNA has dG of +0.8 kcal/mol [9]. Single point mismatched base pairing has subtle negative effect on DNA hybridization if the DNA fragment is of certain length (e.g. >40 nts as a practical lower limit). Even with one or several unmatched nucleotides, two complementary single DNA strands can effectively form DNA double helix as complete matched sequences do under common conditions (FIG. 1). In one aspect, this disclosure relates to the mechanism of reversible DNA base pairing to form heterozygous DNA double strands containing interior loop(s) caused by mismatched base pairing.

In one embodiment, activities of mismatch-repairing DNA enzymes are used to enhance the apparent ratio between two DNA species with one or several mismatched nucleotides. Mismatched base pairing is a common phenomenon in living cells. DNA mismatching may occur by mistakes made by DNA polymerases during DNA replication or caused by environmental factors. To protect itself, cell has developed multiple systems to detect and to remove the mismatched base pairing. All known repairing systems apply either mismatched DNA endonucleases or single strand nucleases, which recognize, cut and repair the mismatched DNA sequence. Examples of such enzymes include, but not limited to, T4 DNA endonuclease, T7 endonuclease, lambda endonuclease, etc. In one embodiment of the current invention, the ability of DNA mismatch-repairing endonucleases and single strand nucleases for digesting DNA strands with nucleotide mismatches is developed into a novel process of amplifying the ratio of different DNA species, whose sequences are mostly the same except for one or a few mismatches. For the convenience of reference, the invented process can be termed “DNA Ratio Alteration by Digest, or DRAD”. Digestion by mismatch repair enzymes has been previously used for detection of mutants or single nucleotide polymorphism (SNP). However, prior applications of the said enzymes employ a process that involves detecting the abundance of DNA fragments generated by enzymatic cutting, or the “split” DNA fragments. Rather than the split DNA fragments, in one embodiment, the current invention relies on the AUGMENTED RATIO between two different DNA species of the intact, uncut target DNA sequence of the original lengths for cellular studies or diagnostics. The said augmented ratio can facilitate the finding of a correlation between DNA (or RNA, which can be reverse transcribed into DNA by methods known in the art) species ratio variation and a disease state.

In another embodiment, the ratio of DNA (or RNA, for simplicity, only DNA is described hereafter) molecules of the same or highly homologous sequence but different lengths, i.e. one DNA molecule that is a portion of a longer DNA molecule, maybe altered by using DNA cutting enzymes that would remove a single-stranded DNA from its 5′ end if the said 5′ end is at a blunt end in pairing with a matching strand, or contains a flap or branched structure. Such enzymes may include, but are not limited to, 5′-3′ exonuclease such as T4 RNaseH (despite is name), lambda exonuclease, T5 exonuclease, Taq 5′ exonuclease, etc [10]. In applying the invented technology, the DNA strand that contains a unique type of the 5′ end in relevance to its matching strand, e.g. blunt as opposed to 5′ overhang, will be preferentially digested, sometimes in the presence of other, helping factors such as single strand DNA binding proteins (SSB) such as the T4 32 protein [11]. Some of the above mentioned enzymes may recognize and digest DNA.DNA or RNA.DNA hybrids, providing an opportunity for analyzing polynucleotide signals composed of either or both of DNA and RNA molecules. The invented process, if carried out in vivo by means of introducing DNA or RNA molecules inside cells in which abnormal polynucleotides exit, can also be used to remove disease-causing and otherwise unwanted RNA or DNA species as a means of therapy.

For practical purposes, the ratio between two RNA molecules or one RNA versus one DNA molecules can always be first converted to DNA versus DNA ratios by reverse transcription, a process well known in the art.

The present disclosure can be used in studying of functions of genes, their effects to cells, tissues, organs, or organisms by correlating DNA sequence variations to phenotypes in general cellular or molecular biology research. The invention can also be applied to clinical diagnostics. For instance, detection of minor DNA species may be used for non-invasive diagnosis of Down's syndrome, Edwards' syndrome, triple X syndrome, etc. The average content of cell free fetal DNA in maternal plasma is low, from about 3% to 5% in some reports to about 10% or somewhat higher in others [1, 2]. The low percentage of fetal DNA in maternal plasma made it impossible or difficult to perform prenatal diagnostics because such low target signal is interfered by maternal DNA, leaving the signal out of the reliable detection range by PCR, MASS analysis, DNA chip array, or other currently available methods for target sequence recognition [3]. Therefore, augmentation of the ratio of abnormal DNA, for instance in the case of Down's syndrome the ratio between the chromosome 21 DNA to other chromosomes in maternal blood samples, is crucial for efficient and reliable diagnosis.

In an additional aspect that the current invention relates to gene expression patterns in the form of different levels of messenger RNAs (mRNAs) or mRNA alternative splicing species (also called isoforms) may be detected and analyzed to study status of cells or onset of diseases. Gene expression profiling by microarrays, high throughput sequencing, reverse-transcription and real-time PCR, etc. has been extensively conducted in many fields of biomedical research. As a particular example, the ratio of certain mRNA species in disease-affected tissues to those in normal tissues may be different. Detection of the ratio change may be used for diagnostic purposes. However, in many cases, detecting such ratio changes or the minor species is difficult due to the fact that the sensitivity of current detection methods are not high enough to generate detectable signal to reflect the slight ratio variations or abundance of minor species.

Probability calculation before and after a preferential DNase enzyme digest: When 2 sets of DNA fragments, which share most of the same sequence and contain one or multiple nucleotide point mutations, are mixed, denatured and annealed, 4 sets of annealed molecules will result: 2 sets of DNA homoduplicates (double-stranded DNA or exactly the same sequence) with the same identity as the input DNA molecules, and 2 sets of heteroduplicates (double-stranded DNA containing one strand each from the 2 input DNA molecules). The possibility for each form can be calculated with following formulas:

P_{(AA′)}={C_{(AA′)}/(C_{(AA′)}+C_{(BB′)})}²

P_{(AB′)}=C_{(AA′)}*C_{(BB′)}/(C_{(AA′)}+C_{(BB′)})²

P_{(A′B)}=C_{(AA′)}*C_{(BB′)}/(C_{(AA′)}+C_{(BB′)})²

P_{(BB′)}={C_{(BB′)}/(C_{(AA′)}+C_{(BB′)})}²
P_{(AA′)}: probability of AA′ combination
P_{(AB′)}: probability of AB′ combination
P_{(A′B)}: probability of A′B combination
P_{(BB′)}: probability of BB′ combination
C: concentration of DNA fragments

When DNA repair enzymes such as T7 endonuclease are mixed with the pool of the above defined 4 sets of DNA duplicates, the heteroduplicate species will be preferentially converted into shorter fragments by cutting at locations surrounding the mismatch(es) (FIG. 1). In consequence, the ratios between the two original DNA species will have been altered, as calculated by the above equations and sample ratio changes summarized in Table 1.

TABLE 1
Examples of Change of Allele Ratios Caused by Mismatch Repair Enzyme Treatment:

| | | input differential | | post treatment (PT) differential | | | differential | Differential |
|---|---|---|---|---|---|---|---|---|
| case | Allele | copy # | (AA′ − BB′)/BB′ | # remained copies | % decrease | (AA′ − BB′)/BB′ | PT/input | (PT − input)/input |
| 1 | AA′ | 100 | | 50 | 50% | | N/A | N/A |
| | BB′ | 100 | 0% | 50 | 50% | 0% | | |
| 2 | AA′ | 101 | | 51 | 49.75% | | 2.01 | 101.00% |
| | BB′ | 100 | 1.00% | 50 | 50.25% | 2.01% | | |
| 3 | AA′ | 103 | | 523 | 49% | | 2.03 | 103.00% |
| | BB′ | 100 | 3.00% | 493 | 51% | 6.09% | | |
| 4 | AA′ | 105 | | 54 | 49% | | 2.05 | 105.00% |
| | BB′ | 100 | 5.00% | 49 | 51% | 10.25% | | |
| 5 | AA′ | 110 | | 58 | 48% | | 2.10 | 110.00% |
| | BB′ | 100 | 10.00% | 48 | 52% | 21.00% | | |
| 6 | AA′ | 120 | | 65 | 45% | | 2.20 | 120.00% |
| | BB′ | 100 | 20.00% | 45 | 55% | 44.00% | | |
| 7 | AA′ | 150 | | 90 | 40% | | 2.50 | 150.00% |
| | BB′ | 100 | 50.00% | 40 | 60% | 125.00% | | |
| 8 | AA′ | 200 | | 133 | 33% | | 3.00 | 200.00% |
| | BB′ | 100 | 100.00% | 33 | 67% | 300.00% | | |
| 9 | AA′ | 250 | | 179 | 29% | | 3.50 | 250.00% |
| | BB′ | 100 | 150.00% | 29 | 71% | 525.00% | | |
| 10 | AA′ | 300 | | 225 | 25% | | 4.00 | 300.00% |
| | BB′ | 100 | 200.00% | 25 | 75% | 800.00% | | |
| 11 | AA′ | 400 | | 320 | 20% | | 5.00 | 400.00% |
| | BB′ | 100 | 300.00% | 20 | 80% | 1500.00% | | |
| 12 | AA′ | 900 | | 810 | 10% | | 10.00 | 900.00% |
| | BB′ | 100 | 800.00% | 10 | 90% | 8000.00% | | |

In one embodiment of the current invention, the augmented ratio of DNA or RNA species may be used to correlate a cellular state or developmental stage. For instance, when a normal cell becomes cancerous, the level of an isoform of a certain gene transcript, AA′, reaches 105 copies versus 100 copies of the reference species BB′, which represents another isoform of the transcript from the said gene (example case 4, Table 1), whereas in normal cells the ratio between AA′:BB′ is 100:100 as in Case 1 of Table 1. The AA′:BB′ ratio change in the said cancerous cells to 5% may not be reliably measured by the current methodologies used in the field such as quantitative RT-PCR or Northern blotting. By using the DRAD procedure, the 5% ratio may be purposefully increased to 10.25% after endonuclease digest (compare post-treatment to input, blue color in Table 1), greatly enhancing the chances for practical measurement of the difference between the two types of cells. When appropriate controls are included in parallel, e.g. measuring ratios between the same transcript isoforms from normal cells and cells, or RNA molecules created by in vitro transcription and are of known concentrations, a correlation can be established with confidence between a slight change of isoform ratio and a particular cellular state.

In another example of the utility of the invented ratio augmentation method, Edwords' syndrome, chromosome 18 trisomy, may be detected by comparing a short homologues region between chromosome 18 and another reference chromosome, for example 22. A fetus with chromosome 18 trisomy would have a higher 18:22 ratio than a normal fetus. However, since fetus contributes only ˜3.4% to 6.2% to the total DNA population in maternal plasma [1], the ratio between a homologues region on chromosome 18 and 22 would be similar to Case 3 and Case 4 in Table 1. To detect a ratio change of such low percentage is extremely difficult, not enough sensitivity can be easily gained to confidently diagnose a disease. By using an enzyme to remove the heteroduplicate species from the pool, however, as described by the current inventors herein, would increase the said ratio, in this hypothetical case by a factor of about 2, putting it into a range that can be reliably detected by MASS or real-time PCR. Even if the percentage of fetal DNA is at the higher estimated 10%-20% [2], as Case 5 or Case 6 illustrated in Table 1, application of the DRAD techniques would still significantly help with the sensitivity and reliability of a non-invasive, prenatal diagnosis.

In another embodiment, ratio between allelic DNA molecules of maternal or fetal origins may be used for non-invasive prenatal diagnosis of autosomal dominant diseases. More specifically, by detecting the presence of fetal-specific paternally inherited mutant alleles in maternal plasma, dominant disease from paternal chromosomes may be detected; absence of fetal-specific paternally transmitted mutant allele can be used to exclude autosomal recessive diseases [2]. However, without using the DRAD technologies, the maternally inherited fetal alleles present in maternal plasma are difficult to discern from the background DNA of the mother because of the overwhelming amount of maternal DNA in the plasma. By preferential digest using mismatch repair enzymes, on the other hand, would significantly change the ratio between fetal and maternal DNA, resulting in improved diagnosis.

Sometimes different alleles manifest their difference in length variations of a particular region in addition to or instead of point mutations. As another important embodiment, the current invention also teaches a method of preferentially removing the shorter DNA strand in an otherwise matched DNA;DNA or DNA:RNA hybrid, which can be used to enhance the probability of discerning fetal DNA.

In one example of this embodiment, DNA isolated from plasma of pregnant women are denatured then renatured, and subjected to T4RNaseH (actually a 5′ exonuclease and flap endonuclease on double-stranded DNA or DNA:RNA). The shorter strand with recessive or blunt 5′ end will be recognized and digested by the said enzyme to remove a few nucleotides, and further digested completely given the right conditions, such as the presence of SSB proteins T4 gene 32 product [11, 12]. The ratio between two alleles, even though one may be of much lower abundance as input, can be dramatically increased, making the difference of being outside or inside of a reliably detectable range with technologies used for diagnosis. Other enzymes that have similar activities that can discriminate homologous but non-identical DNA molecules [10] can be used in the described method of the current invention and included herein by reference. It is also known in the art that there is size discrepancy in general between maternal and fetal cell-free DNA populations [3], it is therefore plausible to use size-biased DNA enzyme to augment the overall ratio of DNA from different sources as a means to enhance the probability of detection.

In yet another embodiment, if the disclosed DRAD process is introduced in vivo, an undesired RNA or DNA species may be removed based on its length difference or point mismatches.

### EXAMPLES

### Example 1-Cloning of T4 DNA Endonuclease and T4 Rnase H

Genomic DNA of T4 phase was purchased from ATCC. PCR primers designed to amply the complete coding regions of T4 endonuclease or T4 RNase H were synthesized at Allele Biotech and used to amply a fragment of predicted size. The fragment was cloned between NdeI and XhoI of the bacterial expression vector pET21a, and the resulting plasmid was used for producing His-tagged recombinant proteins in the BL21 strain of E. coli. These enzymes and T7 endonuclease were also purchased from New England Biolabs (NEB).

### Example 2—Preparation of DNA Fragments for Endonuclease Treatment

This is an exemplary assay to remove heteroduplex DNA that contain a single mismatched base pair while keeping the homoduplicate double stranded (ds)-DNA. The DNA fragments for this experiment were created by PCR reactions. Exemplary test DNA fragment was chosen to be about 200 bp, however, as used herein, suitable sizes can range from about 100 bp to about 1,000 bp, a size range covering most commonly known plasma DNA fragments and exons as detection targets. Wild type and a mutant fluorescent gene Lancelet YFP (LanYFP for short) were used as PCR templates. Wild type LanY gene contains a BamH I recognition site GGATCC and the mutant has a point mutation of the first C to T at the BamH I site (change from GGATCC to GGATTC). A 228 bp DNA fragment was amplified from wild and mutant LanY gene with the BamH I site in the middle of the fragment:

(SEQ ID NO. 1)
ttcaacggtgtggactttgacatggtgggtcgtggcaccggcaatccaa
atgatggttatgaggagttaaacctgaagtccaccaagggtgccctcca
gttctccccctggatTctggtccctcaaategggtatggcttccatcag
tacctgcccttccccgacgggatgtcgcctttccaggccgccatgaaag
atggctccggataccaagtccatcgcacaatg

Plasmid (pCR4-bIFP-Y3) carrying the wild LanY was linearized with Xho I and the mutant plasmid (LanY FPC EC #2) was linearized with Bgl II.

Forward primer LanYEndoF (ttcaacggtgtggac) (SEQ ID NO. 2) and reverse primer LanYEndoR (cattgtgcgatggac) (SEQ ID NO. 3) were synthesized and used to amply the said 228 bp fragment of the LanY gene with BamH I site. PCR was performed in 50 ul reaction with Allele Biotech's PCR master mix at 94 C 30 sec, 48 C 30 sec, 72 C 20 sec for 35 cycles. Multi-reactions were set for each genotype and identical PCR products were pooled and purified with Allele PCR easy column. Exemplary PCR reaction components and reaction conditions are listed in table 2.

TABLE 2
PCR reaction conditions

| | | cat | lot | Stock | final | amount |
|---|---|---|---|---|---|---|
| reagent | manufacturer | # | # | conc | conc | (1rxn) |
| H2O | | | | | | 18 |
| 2X master Mix | Allele | ABP-PP- | 10020 | | | 25 |
| | | MM029 | | | | |
| LanYEndoF(uM) | Allele | Dec. 28, | | 10 | 0.2 | 1 |
| | | 2009 | | | | |
| LanYEndoR(uM) | Allele | Dec. 28, | | 10 | 0.2 | 1 |
| | | 2009 | | | | |
| DNA | Dec. 28, | DIGESED | | | | 5 |
| | 2009 | | | | | |
| total vol (ul) | | | | | | 50 |

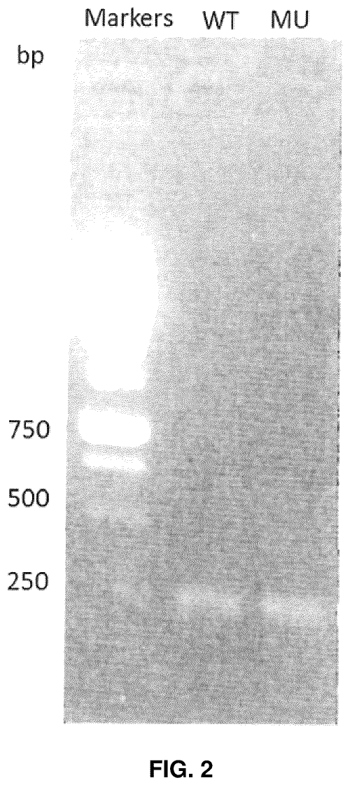

Amplification was double-checked by loading 5 ul PCR product in 1.5% agarose. The target DNA fragments were observed to be specifically amplified (FIG. 2).

### Example 3—T7 DNA Endonuclease Digestion

As an example, T7 Endonuclease I (interchangeably referred to as T7 Endonuclease), which recognizes and cleaves imperfectly matched DNA, cruciform DNA structures, holiday structures or junctions, heteroduplex DNA and more slowly, nicked double stranded DNA was used. The cleavage site is at first, second or third phosphodiester bond that is 5′ to the mismatch. The endonuclease protein is the product of T7 gene3 [13].

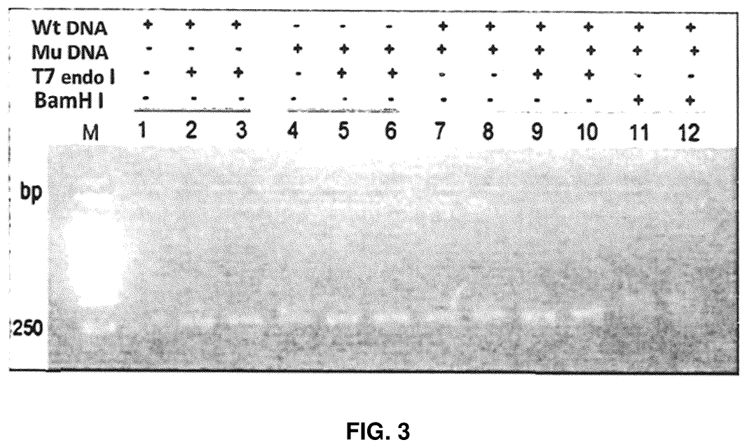

As a structure-selective enzyme, T7 endonuclease I acts on a variety of substrates with different specific activities. To keep the consistence of the substrate, the above PCR amplified DNA fragments of fluorescent gene LanY was used for the titration of digestion conditions. Next, to form the mismatched DNA duplex, wild type and mutant PCR DNA fragments were mixed. DNA double strands were denatured at 94 C for 5 minutes followed by 1 minute of annealing at 60 degree C. Since only one nucleotide is different between the wild type and the mutant DNA fragments in this example, and the mismatch is in the middle with about 100 nucleotides on either side, pairing between the wild type and the mutant DNA should be equal to pairing between identical DNA fragments. The digestion was carried out at 20 C and little or no digestion was observed (FIG. 3). Unexpectedly, the addition of T7 endonuclease I enhanced the pairing of DNA fragments.

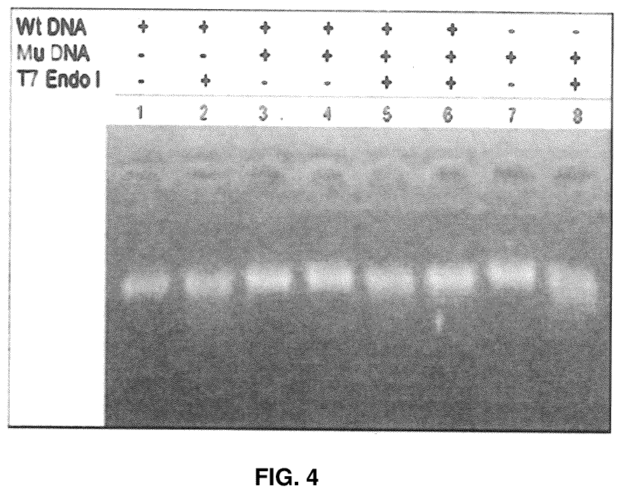

To analyze the apparent non-specific cutting by T7 endonuclease I, we tested 30 minutes' digestions at 20 C, 25 C, 28 C and 43 C with homoduplex DNA. We then set the digestion temperature between 20 C and 28 C with the exemplary digestion time set to 2 hours with fixed incubation temperature at 25 C. FIG. 4 shows the exemplary digestion under these conditions. In the heteroduplex tests (lanes 3-6), addition of T7 endonuclease I decreased the heteroduplex DNA. The decrease of heteroduplex is clearly different from non-specific cutting of homoduplex (lane 8), which produced a smeared DNA band.

### Example 4—Removal of Mismatched DNA by T7 Endonuclease I

As a demonstration of utility, we set up the reactions that contained both wild type and mutant DNA to demonstrate that the mismatched DNA can be removed. The ratio of wild type and mutant DNA in the three groups are 3/2, 2.5/2.5, and 2/3, respectively. Each group of DNA was aliquoted to separate tubes after denature and annealing. This procedure can reduce the possibility of ratio variation from tube to tube. 35 ng DNA was used in each reaction in 10 ul. The reaction settings were listed in table 3.

The digested DNA from each tube was extracted with phenol/chloroform and was precipitated with ethanol. DNA pellet was suspended in 50 ul H2O. As the total amount of DNA in each reaction is 35 ng, the purified DNA was less than 0.7 ng/ul.

TABLE 3
Mismatched DNA setting. Reaction volume = 10 ul.

| tube | WT/Mu | WT/Mu | wt | | mutant | | enzyme | 50 | 25C |
|---|---|---|---|---|---|---|---|---|---|
| # | input | post cut | ng | ul | ng | ul | unit | minutes | hours |
| 1 | 3/2 | 3/2 | 21 | 4.3 | 14 | 3.2 | 0 | 20 | 2 |
| 2 | 3/2 | 3/2 | 21 | 4.3 | 14 | 3.2 | 0 | 20 | 2 |
| 3 | 3/2 | 3/2 | 21 | 4.3 | 14 | 3.2 | 0 | 20 | 2 |
| 4 | 3/2 | 2/1 | 21 | 4.3 | 14 | 3.2 | 5 | 20 | 2 |
| 5 | 3/2 | 2/1 | 21 | 4.3 | 14 | 3.2 | 5 | 20 | 2 |
| 6 | 3/2 | 2/1 | 21 | 4.3 | 14 | 3.2 | 5 | 20 | 2 |
| 7 | 2.5/2.5 | 2.5/2.5 | 17.5 | 3.6 | 17.5 | 4.0 | 0 | 20 | 2 |
| 8 | 2.5/2.5 | 2.5/2.5 | 17.5 | 3.6 | 17.5 | 4.0 | 0 | 20 | 2 |
| 9 | 2.5/2.5 | 2.5/2.5 | 17.5 | 3.6 | 17.5 | 4.0 | 0 | 20 | 2 |
| 10 | 2.5/2.5 | 2.5/2.5 | 17.5 | 3.6 | 17.5 | 4.0 | 5 | 20 | 2 |
| 11 | 2.5/2.5 | 2.5/2.5 | 17.5 | 3.6 | 17.5 | 4.0 | 5 | 20 | 2 |
| 12 | 2.5/2.5 | 2.5/2.5 | 17.5 | 3.6 | 17.5 | 4.0 | 5 | 20 | 2 |
| 13 | 2/3 | 2/3 | 14 | 2.9 | 21 | 4.8 | 0 | 20 | 2 |
| 14 | 2/3 | 2/3 | 14 | 2.9 | 21 | 4.8 | 0 | 20 | 2 |
| 15 | 2/3 | 2/3 | 14 | 2.9 | 21 | 4.8 | 0 | 20 | 2 |
| 16 | 2/3 | 1/2 | 14 | 2.9 | 21 | 4.8 | 5 | 20 | 2 |
| 17 | 2/3 | 1/2 | 14 | 2.9 | 21 | 4.8 | 5 | 20 | 2 |
| 18 | 2/3 | 1/2 | 14 | 2.9 | 21 | 4.8 | 5 | 20 | 2 |

The treated DNA of homoduplicate or heteroduplicate was further analyzed by qPCR and DNA MASSArray for detection of ratio changes.

### Example 5. Plasma DNA Detection

Plasma Separation from Whole Blood and Cell Free Plasma DNA Isolation:

Whole blood was centrifuged at 1,600×g for 10 minutes at 4 C with Brake OFF. Supernatant was transferred to a new tube without disturbing lower level of buffy coat and red blood cells. The sample was centrifuged at 16,000×g for 10 minutes at 4 C with brakes ON. Supernatant was separated from plasma.

Extract Plasma DNA with DSP Blood Mini Kit (Qiagen)

Denature and annealing of DNA fragments (DNA amount can vary from pg to ug depending on target sequence and PCR efficiency):

Reaction buffer was added to DNA solution and DNA was denatured at 94 C for 5 minutes.

Denatured DNA was cooled to 60 C and kept at the temperature for 30 seconds. Then the solution was slowly cooled to room temperature or to 37 C.

Endonuclease Digestion:

In 10 ul reaction, 5 units of T7 Endonuclease I (or other Endonuclease) was added and incubated at 25 C for 2 hours.

100 ul H2O was added to dilute the reaction and 100 ul of phenol-chloroform was added. The mixture was shaken vigorously for 15 seconds. Spun at 10,000 rpm for 5 minutes, then transferred the supernatant to a new tube and 1/10 volume of 5M NaCl was added. 2 volume of 100% ethanol was added. The sample was mixed well and precipitated at −20 C for at least 2 hours. Sample was spun at 15,000 rpm in a bench-top centrifuge for 10 minutes to precipitate DNA. The DNA pellet was washed with 75% ethanol twice.

DNA was suspended in appropriate volume of H2O. The purified DNA was stored at −20 C or applied directly for PCR

Ratio Detection of Digested DNA:

PCR reaction was set up with appropriate primers PCR product was treated with shrimp alkaline phosphatase (SAP) to remove unincorporated dNTPs. SAP was inactivated at 85° C. for 5 min.

Primer extension reaction was then carried out exactly per Sequenom standard procedures. Concentration of extension primers was adjusted according to the efficiency in a multiplex reaction from 0.84 uM to 1.57 uM.

Extension reaction was desalted with Clean Resin and re applied cleaned extension product to SpectroCHIP.

TABLE 4
MassArray analysis data:

| Sample | Well | | | | |
|---|---|---|---|---|---|
| Id | Position | Area 1 | Area 2 | Height 1 | Height 2 |
| S1 | A01 | 88.687 | 76.2961 | 13.9132 | 11.1626 |
| S1 | A02 | 90.794 | 85.9326 | 14.6781 | 11.6802 |
| S1 | A03 | 106.021 | 87.3437 | 14.5936 | 11.6922 |
| S1 | A04 | 93.369 | 87.0473 | 14.006 | 11.9836 |
| S2 | A05 | 58.4576 | 49.7899 | 9.85492 | 6.89126 |
| S2 | A06 | 38.8637 | 36.0916 | 5.58039 | 4.42593 |
| S2 | A07 | 102.189 | 97.178 | 16.32 | 12.574 |
| S2 | A08 | 143.373 | 101.024 | 23.5479 | 15.5733 |
| S3 | A09 | 72.8607 | 66.8063 | 10.8361 | 7.71472 |
| S3 | A10 | 47.23 | 39.8713 | 6.62988 | 5.08486 |
| S3 | A11 | 77.2735 | 65.6137 | 11.6333 | 9.1522 |
| S3 | A12 | 40.949 | 39.0853 | 5.94988 | 5.23816 |
| S4 | B01 | 99.1584 | 84.2435 | 15.5301 | 12.4736 |
| S4 | B02 | 216.288 | 164.58 | 29.671 | 21.8031 |
| S4 | B03 | 223.306 | 183.584 | 31.1102 | 23.8683 |
| S4 | B04 | 176.171 | 158.443 | 26.6102 | 19.5604 |
| S5 | B05 | 81.7038 | 64.8016 | 11.5534 | 8.4375 |
| S5 | B06 | 77.1404 | 63.4741 | 10.9067 | 7.74411 |
| S5 | B07 | 72.5349 | 60.1771 | 11.9668 | 8.55569 |
| S5 | B08 | 91.6608 | 71.6867 | 13.5712 | 10.0342 |
| S6 | B09 | 138.917 | 124.017 | 20.9799 | 15.967 |
| S6 | B10 | 137.877 | 103.832 | 20.3732 | 13.8378 |
| S6 | B11 | 122.63 | 98.225 | 18.9088 | 12.8767 |
| S6 | B12 | 112.732 | 86.4402 | 15.7737 | 10.8053 |
| S7 | C01 | 87.9816 | 112.16 | 12.2873 | 14.1874 |
| S7 | C02 | 69.5783 | 95.4353 | 12.4662 | 13.4046 |
| S7 | C03 | 159.107 | 202.448 | 23.6355 | 27.5343 |
| S7 | C04 | 107.76 | 136.441 | 17.3478 | 18.5629 |
| S8 | C05 | 129.769 | 166.601 | 18.198 | 21.2101 |
| S8 | C06 | 42.818 | 57.1315 | 6.95701 | 7.19527 |
| S8 | C07 | 128.039 | 159.321 | 18.5663 | 20.1974 |
| S8 | C08 | 81.9182 | 102.709 | 12.8918 | 14.0348 |
| S9 | C09 | 152.067 | 182.497 | 21.7602 | 24.7489 |
| S9 | C10 | 94.895 | 107.862 | 14.2852 | 15.6196 |
| S9 | C11 | 112.993 | 143.883 | 17.1694 | 20.3335 |
| S9 | C12 | 71.4614 | 92.327 | 12.0423 | 12.8845 |
| S10 | D01 | 127.502 | 163.9 | 16.3556 | 18.1871 |
| S10 | D02 | 112.524 | 130.456 | 16.6709 | 18.1057 |
| S10 | D03 | 155.337 | 178.512 | 23.3953 | 24.5838 |
| | d04 | | | | |
| S11 | D05 | 79.6294 | 96.0474 | 12.9984 | 14.1838 |
| S11 | D06 | 128.622 | 159.388 | 19.7016 | 21.9034 |
| S11 | D07 | 124.236 | 156.821 | 18.1183 | 19.8507 |
| S11 | D08 | 83.9082 | 104.165 | 13.6396 | 13.5192 |
| S12 | D09 | 118.594 | 150.741 | 16.7771 | 19.1427 |
| S12 | D10 | 138.458 | 163.06 | 20.8198 | 22.2207 |
| S12 | D11 | 62.7942 | 83.25 | 9.18808 | 10.8481 |
| S12 | D12 | 85.5801 | 94.5924 | 13.3729 | 13.9136 |
| S13 | E01 | 76.5551 | 128.709 | 10.4426 | 17.2635 |
| S13 | E02 | 104.839 | 180.434 | 16.1109 | 25.1802 |
| S13 | E03 | 104.362 | 175.608 | 15.528 | 24.2459 |
| S13 | E04 | 56.5538 | 96.789 | 8.87782 | 13.8617 |
| S14 | E05 | 54.4611 | 97.5374 | 8.42612 | 13.8802 |
| S14 | E06 | 66.4348 | 119.41 | 9.57978 | 16.662 |
| S14 | E07 | 62.1042 | 111.236 | 11.12 | 17.6068 |
| S14 | E08 | 62.1004 | 126.81 | 10.1385 | 15.9608 |
| S15 | E09 | 83.2372 | 157.726 | 13.2578 | 22.1477 |
| S15 | E10 | 65.8953 | 119.502 | 9.86454 | 15.9524 |
| S15 | E11 | 63.6662 | 106.564 | 9.25928 | 15.0123 |
| S15 | E12 | 57.6001 | 103.713 | 8.90821 | 14.0559 |
| S16 | F01 | 48.7503 | 89.8091 | 7.94125 | 12.4876 |
| S16 | F02 | 78.4807 | 147.734 | 12.4781 | 21.0728 |
| S16 | F03 | 114.419 | 208.878 | 16.3383 | 28.4658 |
| S16 | F04 | 90.1922 | 163.627 | 13.7824 | 22.73 |
| S17 | F05 | 69.4267 | 128.403 | 9.93251 | 16.5946 |
| S17 | F06 | 77.6288 | 155.086 | 11.9031 | 20.6174 |
| S17 | F07 | 50.1061 | 94.6514 | 7.28247 | 12.4909 |
| S17 | F08 | 67.046 | 122.762 | 10.5017 | 16.391 |
| S18 | F09 | 97.1996 | 169.241 | 15.138 | 25.091 |
| S18 | F10 | 64.7724 | 122.237 | 9.50873 | 15.6857 |
| S18 | F11 | 83.7173 | 148.072 | 13.4204 | 19.5273 |
| S18 | F12 | 81.85 | 147.235 | 11.7658 | 19.1464 |
| WT 2.5 pg | G01 | 269.077 | 0 | 44.497 | 0 |
| WT 2.5 pg | G02 | 346.074 | 0 | 48.0882 | 0 |
| WT 2.5 pg | G03 | 322.448 | 0 | 46.1045 | 0 |
| WT 5 pg | G04 | 546.259 | 0 | 75.9023 | 0 |
| WT 5 pg | G05 | 366.835 | 0.633926 | 54.5466 | 0.256249 |
| WT 5 pg | G06 | 334.942 | 0.197353 | 45.4865 | 0.023179 |
| WT 10 pg | G07 | 336.153 | 0.803077 | 51.4608 | 0.094333 |
| WT 10 pg | G08 | 280.512 | 0 | 41.3011 | 0 |
| WT 10 pg | G09 | 278.686 | 0 | 46.2466 | 0 |
| ddH2O | G10 | 163.993 | 0 | 25.0982 | 0 |
| ddH2O | G11 | 322.786 | 0 | 47.3221 | 0 |
| ddH2O | G12 | 0 | 114.486 | 0 | 15.2223 |
| MU 5 pg | H01 | 0 | 269.653 | 0 | 35.9887 |
| MU 5 pg | H02 | 0.104613 | 289.389 | 0.012387 | 43.8786 |
| MU 5 pg | H03 | 0 | 470.063 | 0 | 60.0272 |
| MU 10 pg | H04 | 0.864825 | 556.377 | 0.102383 | 74.3 |
| MU 10 pg | H05 | 0 | 327.555 | 0 | 43.9229 |
| MU 10 pg | H06 | 0 | 290.499 | 0 | 38.1915 |
| MU 20 pg | H07 | 0 | 252.085 | 0 | 35.8555 |
| MU 20 pg | H08 | 0 | 320.316 | 0 | 44.5909 |
| MU 20 pg | H09 | 0 | 348.494 | 0 | 47.0052 |
| empty | H10 | 0.542239 | 0.49365 | 0.064198 | 0.057983 |
| empty | H11 | 0 | 476.982 | 0 | 61.1689 |
| empty | H12 | 0 | 661.757 | 0 | 90.3646 |
| ddH2O | K21 | 0.548966 | 0.759291 | 0.22852 | 0.278419 |
| ddH2O | K22 | 0.427825 | 0.386846 | 0.050625 | 0.138793 |
| ddH2O | K23 | 0 | 0.925902 | 0 | 0.10868 |
| ddH2O | K24 | 0 | 0 | 0 | 0 |
| empty | L21 | 0.49077 | 0.649147 | 0.05807 | 0.12975 |
| empty | L22 | 3.79123 | 1.81178 | 0.903541 | 0.421311 |
| empty | L23 | 0 | 2.18341 | 0 | 0.376668 |
| empty | L24 | 0.274873 | 0 | 0.032524 | 0 |

TABLE 5
Ratios of input samples and treatment with the endonucleases:

| | Wt/Mu | enzyme |
|---|---|---|
| tube # | input | unit |
| 1 | 3/2 | 0 |
| 2 | 3/2 | 0 |
| 3 | 3/2 | 0 |
| 4 | 3/2 | 5 |
| 5 | 3/2 | 5 |
| 6 | 3/2 | 5 |
| 7 | 2.5/2.5 | 0 |
| 8 | 2.5/2.5 | 0 |
| 9 | 2.5/2.5 | 0 |
| 10 | 2.5/2.5 | 5 |
| 11 | 2.5/2.5 | 5 |
| 12 | 2.5/2.5 | 5 |
| 13 | 2/3 | 0 |
| 14 | 2/3 | 0 |
| 15 | 2/3 | 0 |
| 16 | 2/3 | 5 |
| 17 | 2/3 | 5 |
| 18 | 2/3 | 5 |

TABLE 6
The Wildtype (Wt) vs Mutant (Mu) ratios were enhanced by the enzyme treatment:

| Sample | (Wt − Mu)/Wt | | group sum | | (Wt − Mu)/Mu | | group sum | |
|---|---|---|---|---|---|---|---|---|
| Id | ave | stdev | ave | stdev | ave | stdev | ave | stdev |
| S1 | 0.126 | 0.075 | 0.144 | 0.106 | 0.109283 | 0.058409 | 0.120 | 0.073 |
| S2 | 0.180 | 0.168 | | | 0.141004 | 0.11135 | | |
| S3 | 0.125 | 0.067 | | | 0.108826 | 0.053688 | | |
| S4 | 0.205 | 0.085 | 0.232 | 0.071 | 0.166999 | 0.057714 | 0.186 | 0.048 |
| S5 | 0.240 | 0.035 | | | 0.193079 | 0.02292 | | |
| S6 | 0.250 | 0.093 | | | 0.196605 | 0.062879 | | |
| S7 | −0.228 | 0.029 | −0.209 | 0. 038 | −0.29625 | 0.050383 | −0.267 | 0.059 |
| S8 | −0.218 | 0.024 | | | −0.27906 | 0.040489 | | |
| S9 | −0.182 | 0.048 | | | −0.22553 | 0.071303 | | |
| S10 | −0.163 | 0.051 | −0.178 | 0.046 | −0.19801 | 0.075915 | −0.220 | 0.067 |
| S11 | −0.192 | 0.015 | | | −0.23727 | 0.023189 | | |
| S12 | −0.176 | 0.067 | | | −0.21996 | 0.097873 | | |
| S13 | −0.411 | 0.007 | −0.438 | 0.031 | −0.69911 | 0.020187 | −0.783 | 0.106 |
| S14 | −0.459 | 0.034 | | | −0.85537 | 0.124465 | | |
| S15 | −0.442 | 0.029 | | | −0.79569 | 0.091365 | | |
| S16 | −0.457 | 0.009 | −0.457 | 0.019 | −0.8411 | 0.029856 | −0.844 | 0.067 |
| S17 | −0.471 | 0.020 | | | −0.89182 | 0.074673 | | |
| S18 | −0.444 | 0.019 | | | −0.79898 | 0.063342 | | |

Comparison of the two sets of ratios revealed that (italicized and bold) the ratio from the same input of (Wt−Mu)/Wt increased from 0.144 to 0.232, whereas (Wt−Mu)/Mu changed from −0.783 to −0.844.

### Example 6— Enzyme Activity Test of T4 Rnase H on Double-Stranded DNA

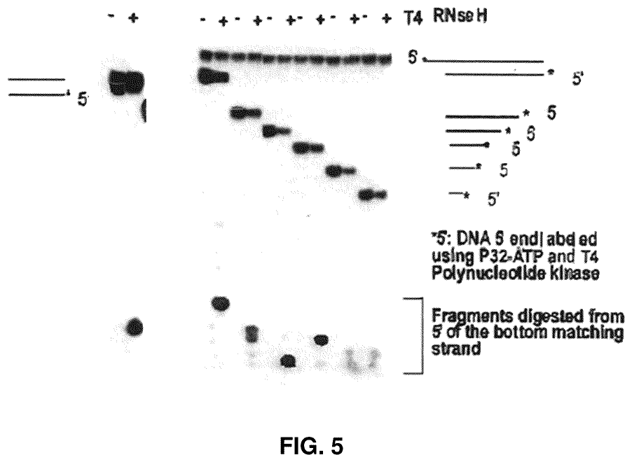

Single-stranded DNA oligos were labeled with P32-ATP and T4 polynuclease kinase (Allele Biotech), annealed to form blunt ends (FIG. 5, left panel) or protruding (FIG. 5, right panel, top strand with light labeling shown on top) or recessive end (FIG. 5, right panel, bottom strands of various lengths shown below the top strand). T4 RNase H (produced at Allele as described in Example 1 or purchased from NEB) was added to digest at room temperature for 40 min. FIG. 5 shows that the DNA strands with blunt end or 5′ recessive end were effectively cut by the enzyme, whereas the strand with pretruding 5′ remained intact. It also shows that the 5′ sequence and/or structure decides the pattern of the released nucleotides from the 5′ end (compare bottom bands of on the right panel, FIG. 5). The blunt or recessive 5′ end containing strand could be further removed completely under favored conditions (not shown). Preferential removal of one of the two matching strands based on length is designed as an embodiment of this invention for altering the ratio between DNA species of different lengths through a denaturing/reannealing process similar to that described in Example 3. The altered ratio can enhance the detection of signals of low abundance DNA or otherwise undiscernable changes of polynucleotides.

### REFERENCES

1. Lo, Y. M., M. S. Tein, T. K. Lau, C. J. Haines, T. N. Leung, P. M. Poon, J. S. Wainscoat, P. J. Johnson, A. M. Chang, and N. M. Hjelm, Quantitative analysis of fetal DNA in maternal plasma and serum: implications for noninvasive prenatal diagnosis. Am J Hum Genet, 1998. 62(4): p. 768-75.
2. Lun, F. M., R. W. Chiu, K. C. Allen Chan, T. Yeung Leung, T. Kin Lau, and Y. M. Dennis Lo, Microfluidics digital PCR reveals a higher than expected fraction of fetal DNA in maternal plasma. Clin Chem, 2008. 54(10): p. 1664-72.
3. Li, Y., B. Zimmermann, C. Rusterholz, A. Kang, W. Holzgreve, and S. Hahn, Size separation of circulatory DNA in maternal plasma permits ready detection of fetal DNA polymorphisms. Clin Chem, 2004. 50(6): p. 1002-11.
4. Chiu, R. W., K. C. Chan, Y. Gao, V. Y. Lau, W. Zheng, T. Y. Leung, C. H. Foo, B. Xie, N. B. Tsui, F. M. Lun, B. C. Zee, T. K. Lau, C. R. Cantor, and Y. M. Lo, Noninvasive prenatal diagnosis of fetal chromosomal aneuploidy by massively parallel genomic sequencing of DNA in maternal plasma. Proc Natl Acad Sci USA, 2008. 105(51): p. 20458-63.
5. Lun, F. M., N. B. Tsui, K. C. Chan, T. Y. Leung, T. K. Lau, P. Charoenkwan, K. C. Chow, W. Y. Lo, C. Wanapirak, T. Sanguansermsri, C. R. Cantor, R. W. Chiu, and Y. M. Lo, Noninvasive prenatal diagnosis of monogenic diseases by digital size selection and relative mutation dosage on DNA in maternal plasma. Proc Natl Acad Sci USA, 2008. 105(50): p. 19920-5.
6. Tabone, T., D. E. Mather, and M. J. Hayden, Temperature switch PCR (TSP): Robust assay design for reliable amplification and genotyping of SNPs. BMC Genomics, 2009. 10: p. 580.
7. Wang, J. and R. A. Hegele, Genomic basis of cystathioninuria (MIM 219500) revealed by multiple mutations in cystathionine gamma-lyase (CTH). Hum Genet, 2003. 112(4): p. 404-8.
8. Lo, Y. M. and R. W. Chiu, Noninvasive prenatal diagnosis of fetal chromosomal aneuploidies by maternal plasma nucleic acid analysis. Clin Chem, 2008. 54(3): p. 461-6.
9. Schildkraut, C. L., J. Marmur, J. R. Fresco, and P. Doty, Formation and properties of polyribonucleotide-polydeoxy-ribonucleotide helical complexes. J Biol Chem, 1961. 236: p. PC2-PC4.
10. Ceska, T. A. and J. R. Sayers, Structure-specific DNA cleavage by 5′ nucleases. Trends Biochem Sci, 1998. 23(9): p. 331-6.
11. Bhagwat, M., L. J. Hobbs, and N. G. Nossal, The 5′-exonuclease activity of bacteriophage T4 RNase H is stimulated by the T4 gene 32 single-stranded DNA-binding protein, but its flap endonuclease is inhibited. J Biol Chem, 1997. 272(45): p. 28523-30.
12. Bhagwat, M. and N. G. Nossal, Bacteriophage T4 RNase H removes both RNA primers and adjacent DNA from the 5′ end of lagging strand fragments. J Biol Chem, 2001. 276(30): p. 28516-24.
13. Sadowski, P. D., Bacteriophage T7 endonuclease. I. Properties of the enzyme purified from T7 phage-infected Escherichia coli B. J Biol Chem, 1971. 246(1): p. 209-16.

1-22. (canceled)

23. A method for preparing a composition comprising an altered ratio of a major allele and a minor allele species from a region of duplexed alleles, said method comprising:
a) providing or having provided a sample comprising double-stranded nucleic acids comprising a region with a major allele and minor allele, wherein the amounts of major and minor allele are present in the sample at a first ratio, wherein the major allele is present in an amount greater than the minor allele,
b) denaturing the sample, thereby forming first single-stranded nucleic acids from the major allele and second single-stranded nucleic acids from the minor allele;
c) reannealing the resulting first and second single-stranded nucleic acids to form major allele homoduplexes and minor allele homoduplexes, and heteroduplexes comprising one single-stranded nucleic acid from the major allele and one single-stranded nucleic acid from the minor allele, the heteroduplexes comprising one or more mismatched base pairs;
d) contacting said major allele homoduplexes and minor allele homoduplexes, and heteroduplexes with an enzyme which cleaves mismatches in duplex nucleic acids to form a composition comprising non-cleaved major allele homoduplexes, non-cleaved minor allele homoduplexes, and cleaved heteroduplexes;
e) performing PCR on said non-cleaved major allele homoduplexes and non-cleaved minor allele homoduplexes to form a composition comprising amounts of major allele double-stranded nucleic acids and minor allele double-stranded nucleic acids present at an altered ratio in the composition.

24. The method of claim 23, wherein said enzyme is a bacteriophage or a eukaryotic enzyme.

25. The method of claim 24, wherein said bacteriophage enzyme is T4 Endonuclease.

26. The method of claim 24, wherein said bacteriophage enzyme is T7 Endonuclease I.

27. The method of claim 23, wherein said enzyme is lambda endonuclease.

28. The method of claim 23, wherein said enzyme is T4 RNAseH.

29. The method of claim 23, wherein the sample is obtained from a eukaryotic cell, a eubacterial cell, a bacterial cell, a mycobacterial cell, a bacteriophage, a DNA virus, or an RNA virus.

30. The method of claim 29, wherein the sample is obtained from a human cell.

31. The method of claim 23, wherein when the one or more mismatched base pairs is from a mutation.

32. The method of claim 31, wherein said mutation is in DNA from subject having a heritable disease or condition.

33. The method of claim 32, wherein the disease or condition is cancer.

34. The method of claim 23, further comprising:
(f) measuring amounts of the major allele double-stranded nucleic acids and minor allele double-stranded nucleic acids in the provided sample and the composition and obtaining the ratio of major allele double-stranded nucleic acids to minor allele double-stranded nucleic acids in the provided sample and the composition, thereby detecting the altered ratio of major and minor nucleic acid species in the composition compared to the provided sample.

35. A method for detecting an altered ratio of a major and minor nucleic acids species from a region of duplexed nucleic acids, said method comprising:
a) providing or having provided a sample comprising double-stranded nucleic acids comprising a region with a major allele and minor allele, wherein the amounts of major and minor allele are present in the sample at a first ratio, wherein the major allele is present in an amount greater than the minor allele,
b) denaturing the sample, thereby forming first single-stranded nucleic acids from the major allele and second single-stranded nucleic acids from the minor allele;
c) reannealing the resulting first and second single-stranded nucleic acids to form major allele homoduplexes and minor allele homoduplexes, and heteroduplexes comprising one single-stranded nucleic acid from the major allele and one single-stranded nucleic acid from the minor allele, the heteroduplexes comprising one or more mismatched base pairs;
d) contacting said major allele homoduplexes and minor allele homoduplexes, and heteroduplexes with an enzyme which cleaves mismatches in duplex nucleic acids to form a composition comprising non-cleaved major allele homoduplexes, non-cleaved minor allele homoduplexes, and cleaved heteroduplexes;
e) performing PCR on said non-cleaved major allele homoduplexes and non-cleaved minor allele homoduplexes to form a composition comprising amounts of major allele double-stranded nucleic acids and minor allele double-stranded nucleic acids present at an altered ratio in the composition;
f) measuring amounts of the major allele double-stranded nucleic acids and minor allele double-stranded nucleic acids in the provided sample and the composition and obtaining the ratio of major allele double-stranded nucleic acids to minor allele double-stranded nucleic acids in the provided sample and the composition, thereby detecting the altered ratio of major and minor nucleic acid species in the composition compared to the provided sample.

36. The method of claim 35, wherein said enzyme is a bacteriophage or a eukaryotic enzyme.

37. The method of claim 35, wherein said bacteriophage enzyme is T4 Endonuclease.

38. The method of claim 35, wherein said bacteriophage enzyme is T7 Endonuclease I.

39. The method of claim 35, wherein said enzyme is T4 RNAseH.

40. The method of claim 35, wherein at least one strand of said duplex nucleic acid is obtained from a eukaryotic cell, a eubacterial cell, a bacterial cell, a mycobacterial cell, a bacteriophage, a DNA virus, or an RNA virus.

41. The method of claim 35, wherein at least one strand of said duplex nucleic acid is obtained from a human cell.

42. The method of claim 35, wherein said mismatch indicates the presence of a mutation.

43. The method of claim 35, wherein said mutation is in DNA from subject having a heritable disease or condition.

44. The method of claim 43, wherein the disease or condition is cancer.
